(19) United States
(12) Patent Application Publication
(10) Pub. No.: US 2016/0059292 A1
(43) Pub. Date: Mar. 3, 2016

(54) BENDING DEVICE

(71) Applicant: Namiki Precision Jewel Co., Ltd., Tokyo (JP)

(72) Inventors: Norikazu Sato, Kuroishi-shi (JP); Tomoyuki Kugo, Kuroishi-shi (JP); Takafumi Asada, Kuroishi-shi (JP)

(21) Appl. No.: 14/470,308

(22) Filed: Aug. 27, 2014

(51) Int. Cl. B21D 11/22; H02K 7/14; A61B 1/005; B21D 7/024

(52) U.S. Cl. CPC B21D 11/22; B21D 7/024; H02K 7/14; A61B 1/0055

(57) ABSTRACT

A bending device to bend a flexible tube from inside electrically includes an electric actuator to drive a movable shaft electrically along the longitudinal direction of the tube in the axial direction and in the rotational direction, and an elongated member of which one end is connected to the movable shaft and the other end is connected to the inner surface of the tube at the location distanced, in the longitudinal direction of the tube, from the connecting point connecting the one end of the elongated member and the movable shaft, the connecting point connecting the one end of the elongated member and the movable shaft being located eccentric to the center of the movable shaft so that the towing direction of the elongated member towed by retreating the movable shaft changes along the circumferential direction by rotation of the movable shaft.

### TECHNICAL FIELD

The present invention relates to a bending device configured to bend a flexible tube from inside by an electric mechanism or the like, particularly to a bending device preferably used for a medical endoscope device or an industrial endoscope device to perform observation, treatment, or the like by the observer inserting the tube in the tubular passage, bending the tube in the intended direction, and aiming a camera, a diagnosis sensor, or the like provided on the distal end of the tube at a portion to be observed or a lesion located further deeper inside the tubular passage.

### RELATED ART

Medical equipment is rapidly progressing. Particularly, a less invasive treatment of a colon and a small intestine using an endoscope is conventionally performed only for treating a colorectal and a region of the colon adjacent to the colorectal (a descending colon and a transverse colon) because a thick tube of the endoscope device is hard to bend so that the tube cannot be inserted into a deep portion of a living body. However, in recent years, it is desired to perform observation of a lesion and less invasive treatment without performing laparotomy, by inserting a flexibly bendable endoscope tube into the deepest portion of the colon.

As an endoscope device for medical treatment described above, for example, an invention is disclosed as described below.

In the invention disclosed in JP 61-106126 A, as illustrated in FIG. 2 and FIG. 3, a bending portion (16) in the distal end side of an endoscope tube (flexible tube: 17) includes therein a plurality of bendable tubular joint pieces (26) connected in an axial direction, four bending wires (30) supported on the circumference of the joint pieces with a space between each other, and wire driving units 31a to 31d (linear motor). By the wire driving unit selectively pulling the bending wire, the bending portion bends in all up-and-down and right-and-left directions.

In the invention disclosed in Japanese Patent No. 2608590, as illustrated in FIG. 3 and FIG. 4, a bending portion (7) in the distal end side of an endoscope tube (flexible tube: 8) includes a plurality of bendable tubular joint pieces (18) connected in an axial direction, four bend control wires (22) supported, circumferentially spaced between each other, in the joint pieces, and wire driving units 23a to 23d (linear motor). By the wire driving unit selectively pulling the bend control wires, the bending portion can be bent in all up-and-down and right-and-left directions.

In the invention disclosed in Japanese Patent No. 2653844, as illustrated in FIG. 1 and FIG. 2, a bending portion (4) in the distal end side of an endoscope tube (flexible portion: 5), a plurality of bend driving wires (19a, 19b) each having an endless-belt-like shape disposed throughout the inside of the bending portion and a controller (2) in the proximal end side of the bending portion, and wire drums (18a, 18b) on which the bend driving wires runs about in the controller are provided. The bending portion can be bent in all up-and-down and right-and-left directions by rotating the wire drums so as to selectively pull the bend driving wires.

However, in any of the prior art described above, a plurality of wires, a plurality of wire driving units (or wire drums) for selectively pulling the wires, or numbers of electric cords or the like for supplying electric power to the wire driving units are required, which likely to results in a large outer diameter of the tube. Moreover, the rigidity of such thick tube and the numbers of wires and electric cords deteriorate flexibility of the tube in the longitudinal direction, which may result in poor bending ability.

Therefore, in the prior art, when a medical endoscope device configured with a sensor (e.g., CCD camera) attached on the distal end of the tube is inserted in a living body as described in FIG. 12, for example, the tube 100 cannot flexibly bend along the winding passage having a plurality of corners, that is, a rectum 111, a sigmoid colon 112, a descending colon 113, a left colic flexure 114, a transverse colon 115, a right colic flexure 116, and the like. For this reason, even though the endoscope device can possibly reach the transverse colon 115, it may be difficult to observe and treat the portion located further deeper than the right colic flexure 116 such as the ascending colon 117 and the deepest portion of the colon, as illustrated in the drawing.

### CITATION LIST

### Patent Literature

Patent Literature 1: JP 61-106126 A

Patent Literature 2: Japanese Patent No. 2608590

Patent Literature 3: Japanese Patent No. 2653844

### SUMMARY OF INVENTION

The present invention is made in view of the problem of the prior art. The object of the present invention is to provide a thin bending device having flexibility allowing the bending device to easily bend.

As a means for solving the problem mentioned above, a bending device configured to bend a flexible tube electrically from inside. The bending device includes an electric actuator configured to drive a movable shaft electrically along the longitudinal direction of the tube in the axial direction and in the rotational direction, and an elongated member of which one of ends is connected to the movable shaft and the other end is connected to the inner surface of the tube at the location distanced, in the longitudinal direction of the tube, from the connecting point connecting the one of ends of the elongated member and the movable shaft. The connecting point connecting the one of ends of the elongated member and the movable shaft is located eccentric to the center of the movable shaft so that the towing direction of the elongated member towed by retreating the movable shaft changes along the circumferential direction by the rotation of the movable shaft.

### BRIEF DESCRIPTION OF DRAWINGS

FIG. 1 is an internal structural drawing illustrating an example of a bending device according to the present invention;

FIG. 2 is a longitudinal cross sectional view of the bending device illustrating a state before bending;

FIG. 3 is a longitudinal cross sectional view of the bending device illustrating a state in which the bending device is bent toward a certain direction;

FIG. 4 is a longitudinal cross sectional view of the bending device illustrating a state in which the bending device is bent toward the other direction;

FIG. 5 is a perspective view illustrating the internal structure of the bending device;

FIG. 6 is a side cross sectional view of the bending device;

FIG. 7 is a longitudinal cross sectional view illustrating an example of the bending device using a plurality of drive units;

FIG. 8 is a schematic drawing of an example of the endoscope device equipped with the bending device;

FIG. 9 is a schematic drawing illustrating the bending device according to the present invention inserted in a body;

FIG. 10 is a longitudinal cross sectional view illustrating another example of the bending device according to the present invention;

FIG. 11 is a longitudinal cross sectional view illustrating another example of the bending device according to the present invention; and

FIG. 12 is a schematic drawing illustrating a conventional bending device inserted in a body.

### DETAILED DESCRIPTION

A first feature of the bending device according to the embodiment is that a bending device configured to bend a flexible tube from inside electrically includes an electric actuator configured to drive a movable shaft electrically along the longitudinal direction of the tube, in the axial direction and in the rotational direction and an elongated member of which one of ends is connected to the movable shaft and the other end is connected to the inner surface of the tube at the location distanced, in the longitudinal direction of the tube, from the connecting point connecting the one of ends of the elongated member and the movable shaft. The connecting point connecting the one of ends of the elongated member and the movable shaft is located eccentric to the center of the movable shaft so that the towing direction of the elongated member towed by retreating the movable shaft changes along the circumferential direction by the rotation of the movable shaft.

According to the configuration, when the elongated member is towed by retreating the movable shaft by the driving force of the electric actuator, the tube is pulled by the elongated member and is bent. When the movable shaft rotates by the driving force of the electric actuator, the towing direction of the elongated member changes along the circumferential direction so that the bending direction of the tube also changes along the circumferential direction.

The second feature of the bending device according to the embodiment is that the bending device includes a connecting member connected to the inner surface of the tube and also connected to the other end of the elongated member, and that the connecting point connecting the other end of the elongated member and the connecting member is located distanced from the inner surface of the tube.

By this configuration, operability to rotate the movable shaft to change the bending direction of the tube in the circumferential direction can be improved.

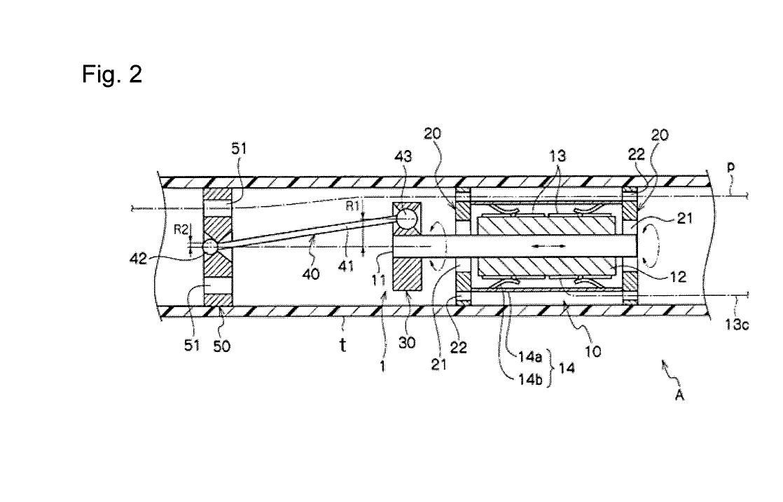

The third feature of the bending device according to the embodiment is that a first radius and a second radius are configured to have a dimensional relationship of first radius>second radius (see FIG. 2), where the first radius is the eccentric radius of the connecting point connecting the one of ends of the elongated member and the movable shaft about the center of the movable shaft and the second radius is the eccentric radius of the connecting point connecting the other end of the elongated member and the connecting member about the center of the movable shaft.

By this configuration, operability to rotate the movable shaft to change the bending direction of the tube along the circumferential direction can further be improved.

In an aspect to further improve the operability and to provide higher productivity, the connecting point connecting the other end of the elongated member and the connecting member is located on the central axis of the movable shaft so as the second radius to be approximately zero. Further preferably, the connecting point connecting the other end of elongated member and the connecting member is located in approximately central portion in the radial direction of the tube so as the second radius to be approximately zero.

The fourth feature of the bending device according to the embodiment is that the elongated member is rotatably and swingably connected to the movable shaft and also rotatably and swingably connected to the connecting member.

By this configuration, a specific structure having high operability to change the bending direction of the tube along the circumferential direction can be provided.

A fifth feature of the bending device according to the embodiment is that the electric actuator includes a vibratable member making contact with the outer periphery of the movable shaft, a piezoelectric element secured on the vibratable member, and an electrode secured on the piezoelectric element and that a plurality of sets of electrodes is arrayed in the circumferential direction and in the axial direction.

In this configuration, when the plurality of sets of electrodes arrayed in the axial direction is electrified in order, an oscillatory wave progressing in the axial direction is generated in the piezoelectric element and in the vibratable member. The vibratable member linearly moves in the axial direction by the oscillatory wave. When the plurality of sets of electrodes arrayed in the circumferential direction is electrified in order, an oscillatory wave progressing in the circumferential direction is generated in the piezoelectric element and in the vibratable member. The movable shaft rotates by the oscillatory wave.

In a preferable aspect to improve transmission efficiency of the oscillatory wave, a plurality of vibratable members is provided around the movable shaft, separated from each other in the circumferential direction.

Further, in an aspect to further improve transmission efficiency of the oscillatory wave, an urging member (e.g., a flat spring or a coil spring) urging the vibratable member in the radial direction thereby pushing the vibratable member against the outer circumferential surface of the movable shaft is included. In this configuration, the urging force of the urging member produces a friction force between the vibratable member and the movable shaft so that the movable shaft stays in the position even after cutting off the electric supply to the electric actuator. This maintains the bent state of the tube without electric supply.

Further, a wiring such as a signal line and a power line is inserted in the gap between the inner circumferential surface of the tube and the vibratable member as required.

A sixth feature of the bending device according to the embodiment is that a recovering member for urging the tube to return to the state before bent is provided.

In this configuration, the tube can rapidly return to the state before bent by the urging force of the recovering member.

The “recovering member” includes configurations such as the configuration urging the tube to return to the state before bent by the shape retaining property of the tube itself and the configuration urging the tube to return to the state before bent by other members.

A seventh feature of the bending device according to the embodiment is that the elongated member is formed with a flexible long body.

In this configuration, when an external force to bend the tube is applied, the tube and the elongated member can flexibly bend without resisting against the external force. This is particularly useful when the bending device is used as a medical device to be inserted in a body (such as an endoscope device).

Further, another preferable feature of the bending device according to the embodiment is that a penetration hole penetrating the movable shaft in the axial direction is provided and that wirings are disposed in the penetration hole.

In this configuration, deterioration in bending ability of the tube caused by the wiring can be lessened.

Further, another preferable feature of the bending device according to the embodiment is that a tubular support member capable of deforming along the bending portion of the tube so as to stay close to, or to contact, the inner circumferential surface of the bent portion of the tube is provided.

In this configuration, forming of wrinkles and folds on the inner circumferential wall of the bending portion that is likely to occur when the tube is bent can be prevented by the tubular support member.

Further, in another preferable aspect, a plurality of drive units each including the electric actuator and the elongated member is included and the plurality of drive units is provided, with a longitudinal gap between each other, inside a single tube. The tube is configured to bend at each of a plurality of longitudinal locations by the drive unit.

In this configuration, the tube can be bent at each of the plurality of longitudinal locations. For example, when the bending device is used as a medical device to be inserted in a body (such as an endoscope), the tube can bend along a plurality of corners or the like in the body.

Now, a preferable examples of the embodiment having these features will be described in detail based on the drawings.

Note that, in the description below, the longitudinal direction of the tube t represents the direction in which the central axis of the tube t extends. The radially outer direction of the tube t represents the radial direction toward the outer side of the tube t (in other words, the centrifugal direction). The radially inner direction of the tube t represents the radial direction toward the inner side of the tube t (in other words, the centripetal direction).

### Embodiment 1

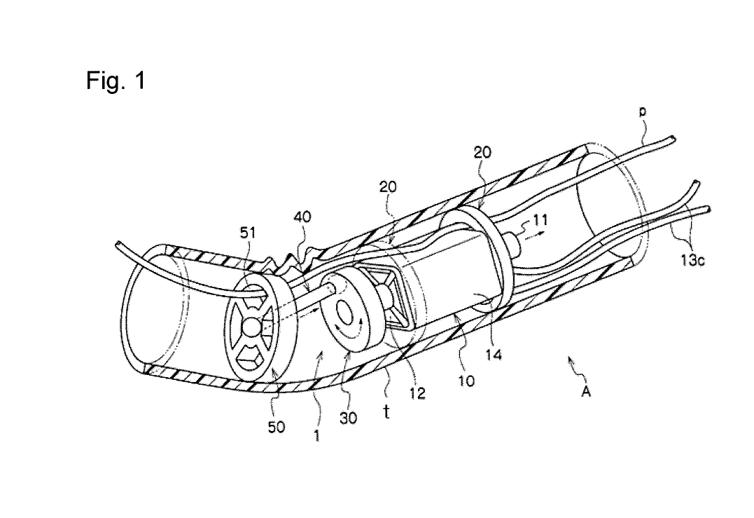

A bending device A illustrated in FIG. 1 to FIG. 6 includes a flexible tube t and a drive unit 1 inserted in the tube t. The bending device A is configured to bend the tube t by the drive unit 1 by electric power.

The tube t may be any tube which can be bent by the force from the drive unit 1, which will be described below, and has shape retaining property (resilience) to return to the original shape (a linear-tubular shape, in the exemplary drawing) when the force from the drive unit 1 is not applied. For example, the tube t is formed of an elastic synthetic resin or a rubber in a cylindrically straight shape.

The drive unit 1 includes an electric actuator 10 configured to drive the movable shaft 11, provided along the longitudinal direction of the tube t, in the axial direction and in the rotational direction by electric power, a secure bracket 20 for securing the electric actuator 10 on the inner circumferential surface of the tube t, an eccentric connecting member 30 secured on the front end (left end, in FIG. 1) of the movable shaft 11, an elongated member 40 of which rear end is connected to the eccentric connecting member 30 at the point eccentric to the movable shaft 11, and a connecting member 50 connected to both the front end of the elongated member 40 and the inner circumferential surface of the tube t.

As illustrated in FIG. 2 to FIG. 6, the electric actuator 10 includes the movable shaft 11 disposed in approximately center of the electric actuator 10, a plurality of vibratable members 12 disposed, separated from each other in the circumferential direction, on the periphery of the movable shaft 11, a piezoelectric unit 13 secured on the outer surface of each vibratable member 12, and a casing member 14 urging the vibratable member 12 in the radially inner direction by the urging member 14b pushing the vibratable member 12 against the outer circumferential surface of the movable shaft 11. When the piezoelectric unit 13 is electrified, the vibration of the vibratable member 12 makes the movable shaft 11 linearly move and rotate.

The movable shaft 11 may be any column-shaped or cylindrical long body in which at least a portion of the cylindrical surface of the long body makes contact with the vibratable member 12 disposed on the outer circumferential surface of the long body. For example, the movable shaft 11 may be formed of a rigid material such as metals.

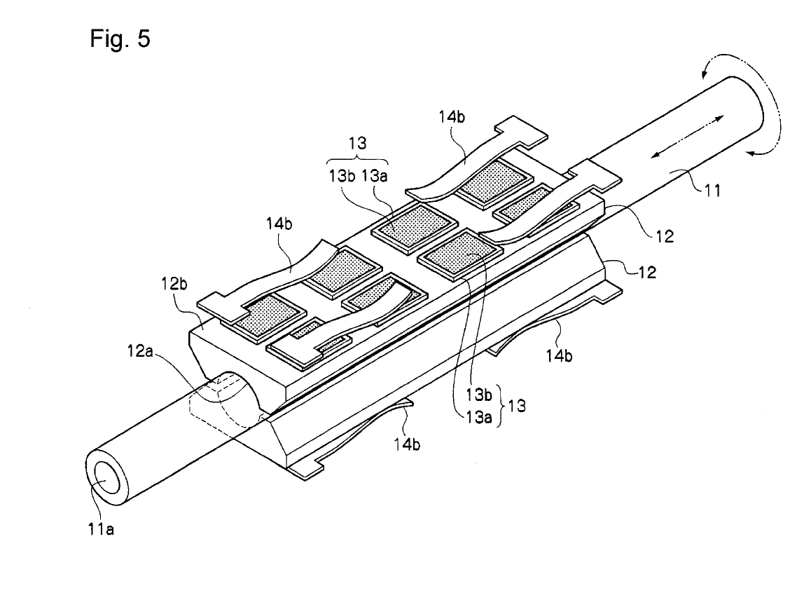

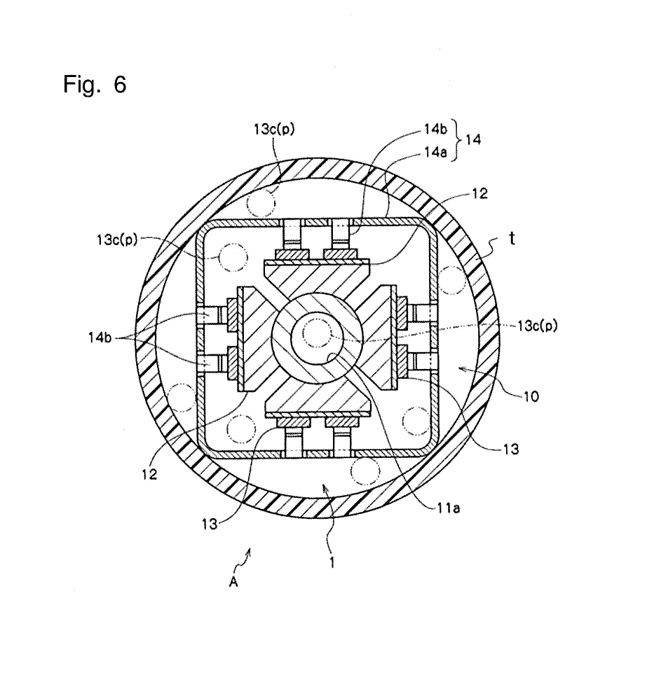

In the preferable example illustrated in the drawing, the movable shaft 11 includes a penetration hole 11a (see FIG. 5 and FIG. 6) penetrating the movable shaft 11 in the axial direction. A wiring such as a signal line and a power line can be inserted in the penetration hole 11a as required.

A plurality of vibratable members 12 is disposed, separated from each other in the circumferential direction, so as to make contact with the outer circumferential surface of the movable shaft 11 from the radially outer direction. Note that, in the example illustrated in FIG. 5, two vibratable members 12 are disposed so as to oppose each other in the radial direction. In an example illustrated in FIG. 6, four vibratable members 12 are disposed, evenly spaced along the circumferential direction.

Each vibratable member 12 is formed of a rigid material such as metals and formed into a long plate-like shape extending along the axial direction of the movable shaft 11. The vibratable member 12 has, in the inner circumferential side thereof, a concave surface 12a corresponding to the outer circumferential surface of the movable shaft 11 and has, in the outer circumferential side thereof, a flat surface 12b allowing the piezoelectric unit 13 to be easily attached thereto.

The concave surface 12a is a curved surface, extending in the axial direction along the outer circumferential surface of the movable shaft 11, having an approximately arc shaped cross section. On the concave surface 12a, a single or a plurality of grooves, elongated protrusions, ruggedness, or the like is provided with a suitable distance in between, as required, so as to efficiently transmit the vibration of the vibratable member 12 to the movable shaft 11.

The flat surface 12b is a flat face parallel to the axial direction of the movable shaft 11. The piezoelectric unit 13 is secured on the surface of the flat surface 12b.

A plurality of piezoelectric units 13 is secured on the flat surface 12b of the vibratable member 12 so as to be arrayed in the axial direction and in the circumferential direction of the movable shaft 11. In the example illustrated in FIG. 5, four piezoelectric units 13 are arrayed in the axial direction and two piezoelectric units 13 are arrayed in the circumferential direction.

Each piezoelectric unit 13 is configured with a piezoelectric element 13a provided with a set of electrodes 13b. To each electrode 13b, a wiring 13c (see FIG. 2) for power supply is connected.

The piezoelectric element 13a has a configuration (referred to as a unimorph, etc.) in which a thin piezoelectric element (including those referred to as a piezoelectric ceramic, a piezoelectric element, an electrostrictive element, piezoelectric resin film, etc.) and a metal plate is bonded with an adhesive (e.g., a conductive thermosetting adhesive). According to the example illustrated in the drawing, the piezoelectric element 13a is composed in a single plate on which surface an electrode 13b is formed in a pattern.

The wiring 13c is electrically connected to the electrode 13b to supply power. As illustrated in FIG. 6, the wiring 13c is introduced along the axial direction of the movable shaft 11 through gaps such as the gap between the vibratable member 12 and the casing member 14 and the gap between the casing member 14 and the tube t.

The piezoelectric unit 13 configured as described above produces vibration of progressive waves in the piezoelectric element 13a by sequentially electrifying the plurality of electrodes 13b with electric power having a predetermined frequency. The vibration is transmitted to the movable shaft 11 via the vibratable member 12, thereby moving the movable shaft 11 in the direction corresponding to the sequential order by which the plurality of electrodes 13b is electrified.

For example, when the plurality of electrodes 13b arrayed in the axial direction of the movable shaft 11 is sequentially electrified from the rear side to the front side, the movable shaft 11 linearly moves forward, and when the plurality of electrodes 13b is sequentially electrified from the front side to the rear side, the movable shaft 11 linearly moves backward.

Further, when the plurality of electrodes 13b arrayed in the circumferential direction of the movable shaft 11 is sequentially electrified in the clockwise direction, the movable shaft 11 rotates clockwise, and when the plurality of electrodes 13b is sequentially electrified in the counterclockwise direction, the movable shaft 11 rotates counterclockwise.

In the above-mentioned aspect, the set of electrodes 13b is provided on the single piezoelectric element 13a. In another example, when the piezoelectric element 13a having a unimorph structure is provided, the aspect having a single piezoelectric element provided with a plurality of sets of electrodes arrayed in a predetermined direction can be provided.

In any of the aspects described above, the number of the electrodes 13b arrayed in the axial direction of the movable shaft 11 is preferably three or more so that the moving direction of the movable shaft 11 can easily be identified. However, depending on a control method, an additional configuration, or the like, the number of the electrodes 13b can be two. Similarly, the number of the electrodes 13b arrayed in the circumferential direction of the movable shaft 11 (the number of electrodes 13b arrayed throughout the plurality of vibratable members 12, according to the example illustrated in the drawing) is also preferably three or more so that the rotational direction of the movable shaft 11 can easily be identified. However, depending on a control method, an additional configuration, or the like, the number of the electrodes 13b can be two.

Further, in another example of the piezoelectric element 13a, a structure other than the unimorph structure (e.g., a bimorph structure) can be employed.

The casing member 14 is integrally configured with a movable shaft 11, a case body 14a having a square-tube shape annularly surrounding the periphery of the vibratable member 12 and the piezoelectric unit 13, and an urging member 14b provided as a flat spring protruding inward from the case body 14a (see FIG. 2 and FIG. 6). The casing body 14a is formed of an elastically bendable material such as a metal material so as to allow the urging member 14b to elastically deform like a flat spring.

In the example illustrated in the drawing, the urging member 14b is formed in a flat spring extending in the axial direction. In another example, the urging member 14b may be formed in a flat spring extending in the circumferential direction. Further, in other examples of the urging member 14b, an aspect configuring the urging member 14b as a flat spring provided separately from the case body 14a, an aspect using a coil spring, or an aspect using an elastic body such as a rubber can be provided.

The secure bracket 20 is an annular member each secured on the front end and the rear end of the casing member 14 so as not to rotate nor advance/retreat. The outer periphery of the secure bracket 20 is secured to the inner circumferential surface of the tube t so as not to rotate nor advance/retreat. The secure bracket 20 may be secured to the casing member 14 and to the tube t by a suitable structure, for example, welding or engaging.

Each secure bracket 20 has, in the central portion thereof, a penetration hole 21 in which the movable shaft 11 is inserted, allowing the movable shaft 11 to rotate and advance/retreat.

Further, near the outer periphery of each secure bracket 20, a penetration hole 22 in which wirings p and 13c such as signal lines and power lines are inserted is provided.

In another example, the secure bracket 20 can be omitted and the corner of the outer surface of the casing member 14 can be secured to the inner circumferential surface of the tube t by pressure welding.

The eccentric connecting member 30 is a disk-shaped member secured to the outer periphery of the front end of the movable shaft 11 so as to integrally rotate and advance/retreat with the movable shaft 11.

The eccentric connecting member 30 is connected to the rear end of the elongated member 40 at the point eccentric to the movable shaft 11.

The elongated member 40 is a long member extending in the longitudinal direction of the tube t. The rear end of the elongated member 40 is rotatably and swingably connected to the eccentric point on the eccentric connecting member 30. The front end of the elongated member 40 is rotatably and swingably connected to the connecting member 50 at the point forwardly distanced from the connecting point to the eccentric connecting member 30 by a predetermined length.

In the drawing, a first radius which is the eccentric radius of the connecting point (the center of the engagement part 43 in FIG. 2) connecting the rear end of the elongated member 40 and the movable shaft 11 about the center of the movable shaft 11 is represented by the symbol R1, and a second radius which is the eccentric radius of the connecting point (the center of the engagement part 42 in FIG. 2) connecting the front end of the elongated member 40 and the connecting member 50 about the center of the movable shaft 11 is represented by the symbol R2. The first radius R1 and the second radius R2 are configured to have a dimensional relationship of R1>R2. Ideally, the bending operation can smoothly be performed by providing the second radius R2 close to zero.

In the preferable example illustrated in FIG. 2, the second radius R2 is approximately zero and the center of the engagement part 42 is provided approximately on the center of the movable shaft 11 and the tube t.

The term “rotatable” means that the elongated member 40 can spin about the axis thereof. The term “swingable” means that the elongated member 40 can swing about the connecting point connecting the elongated member 40 and the eccentric connecting member 30.

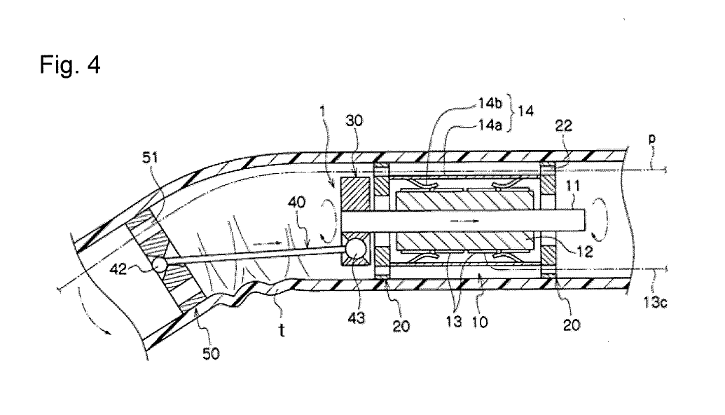

For example, in the preferable example illustrated in FIG. 2 and FIG. 4, the connecting structure is configured with the spherical inner surface holding the spherical engagement part 43 on the rear end of the elongated member 40.

To describe in detail, the elongated member 40 includes a flexible long main body 41, the spherical engagement part 43 integrally connected to the rear end of the main body 41, and a spherical engagement part 42 integrally connected to the front end of the main body 41.

The main body 41 may be any flexible long body. In the example, a metal wire is used. In another example of the main body 41, a synthetic resin wire or a string having a suitable tension strength can be used.

Each of the front and rear engagement parts 42 and 43 is formed in a spherical shape with a hard material such as metals and synthetic resins.

The connecting member 50 is an approximately disk-shaped member. The connecting member 50 is secured to the inner circumferential surface of the tube t so as not to advance/retreat nor rotate and is rotatably and swingably connected to the engagement part 42 on the front end of the elongated member 40.

The connecting member 50 may be connected to the tube t by adhering, engaging, or the like.

The connecting member 50 may be connected to the elongated member 40 by, for example, forwardly inserting the main body 41 of the elongated member 40 through the connecting member 50 and receiving the spherical engagement part 42 on the front end of the elongated member 40 by the conical surface of the connecting member 50 so as to allow rotation and swinging of the elongated member 40, as illustrated in FIG. 2 to FIG. 4.

The connecting point connecting the connecting member 50 and the elongated member 40 is located in radially inner side than the connecting point connecting the rear end of the elongated member 40 and the movable shaft 11 (specifically, the eccentric connecting member 30). Specifically in the example illustrated in the drawing, the connecting point connecting the connecting member 50 and the elongated member 40 is located approximately in the center of the connecting member 50.

Near the outer periphery of the connecting member 50, a penetration hole 51 is provided approximately parallel to the direction of the central axis of the tube t. A wiring p such as a signal line and a power line is inserted in the penetration hole 51. The wiring p can be provided as an electric cord for transmitting electric signal or supplying power source, or as an optical fiber cable for transmitting an optical signal, or the like to a device (e.g., a sensor or a CCD camera) in the tube t.

Now, a distinctive effect of the bending device A configured as described above will be described.

In the initial state in which the drive unit 1 is not electrified, the bending device A is kept in an approximately linear shape as illustrated in FIG. 2.

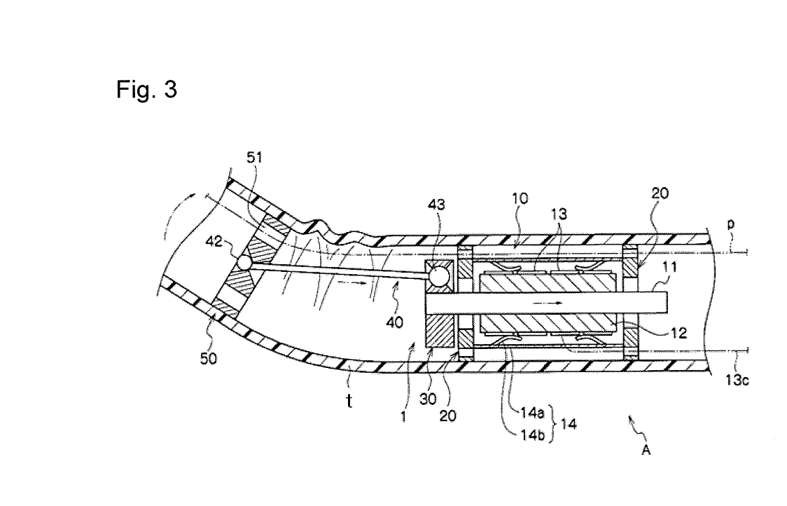

From the initial state, when the movable shaft 11 retreats by sequentially electrifying the plurality of electrodes 13b arrayed in the axial direction of the drive unit 1 from the front side to the rear side, the connecting member 50 is towed toward the drive unit 1 by the elongated member 40 as illustrated in FIG. 3, thereby bending the portion of the tube t between the connecting member 50 and the electric actuator 10. The bending angle in this state can be controlled as desired by controlling the retreat distance of the movable shaft 11 (specifically, controlling the time of electrifying the piezoelectric unit 13).

The bent state is maintained by friction between the vibratable member 12 and the movable shaft 11 even after cutting off the electric supply to the drive unit 1. That is, even though the tube t has elastic resilience, because the vibratable member 12, urged by the urging member 14b, is pushed against the outer circumferential surface of the movable shaft 11, the movable shaft 11 will not advance by the resilience even after cutting off the electric supply to the drive unit 1, so that the bent state of the tube t can be maintained.

When returning the tube t to the original state (linear-tubular shape, as illustrated in FIG. 2), the plurality of electrodes 13b arrayed in the axial direction of the drive unit 1 is sequentially electrified from the rear side to the front side. In this manner, the elongated member 40 advances along with the movable shaft 11 to release tension, thereby allowing the tube t to return to the original approximately linear shape by the elastic resilience (shape retaining property).

As illustrated in FIG. 4, when the bending device A is to be bent toward the opposite direction, first, from the initial state (linear-tubular shape) of the bending device A, the plurality of electrodes 13b arrayed in the circumferential direction of the drive unit 1 is sequentially electrified to rotate the movable shaft 11 by a predetermined amount. Then along with rotation of the eccentric connecting member 30 integrated with the movable shaft 11, the rear end of the elongated member 40 spinningly revolves about the central axis of the movable shaft 11 to change the circumferential location. The motion is smoothly performed without sticking, because the spherical engagement parts 42 and 43 on the front and rear ends of the elongated member 40 spinningly swing (may also be described as: make precession movement) and also the elongated member 40 itself flexibly deforms.

Note that, the amount of revolution of the rear end of the elongated member 40 can continuously be controlled throughout the entire circumference by controlling the amount of rotation of the movable shaft 11 (specifically, by controlling the time of electrifying the plurality of piezoelectric units 13 arrayed in the circumferential direction).

After the rotation, when the movable shaft 11 retreats by sequentially electrifying the plurality of electrodes 13b arrayed in the axial direction of the drive unit 1 from the front side to the rear side, the connecting member 50 is towed toward the drive unit 1 by the elongated member 40, as illustrated in FIG. 4, thereby bending the portion of the tube t between the connecting member 50 and the drive unit 1 toward the direction different from the direction illustrated in FIG. 3 (direction different by 180 degrees, as illustrated in FIG. 4). The bending angle in this state can be controlled as desired by controlling the retreat distance of the movable shaft 11 (specifically, controlling the time of electrifying the piezoelectric unit 13).

As described above, the bent state is maintained by friction between the vibratable member 12 and the movable shaft 11 even after cutting off the electric supply to the drive unit 1. When returning the tube t to the original state (linear-tubular shape, as illustrated in FIG. 2), the plurality of electrodes 13b arrayed in the axial direction of the drive unit 1 is sequentially electrified from the rear side to the front side.

Note that, from the bent state illustrated in FIG. 3, the tube t can be bent toward the opposite direction as illustrated in FIG. 4 without returning to the original linear-tubular shape, by rotating the movable shaft 11.

Accordingly, the bending device A can bend or straighten the tube t throughout the entire 360-degree range as desired and can also control the bending angle of the bending portion as desired.

Moreover, the structure does not require a plurality of wires, a plurality of drive units, or the like so that the number of wirings 13c inserted in the tube t to supply power is relatively small. As in the example illustrated in the drawing, it can be configured with only one elongated member 40 and a single drive unit 1. Therefore, the outer diameter of the tube t can be made relatively small so as to configure the bending device A thin and also to allow the tube t to bend flexibly.

Further, since the elongated member 40 is formed of a flexible material, the tube t can be bent and deformed by an external force. For example, when the bending device A is used for a medical or an industrial endoscope device, since the tube t can flexibly bend and deform by making contact with the inner wall of a tubular passage, the tube t can be inserted into the deep portion of the thin tubular passage having bent portions by bending the tube t as desired by electric operation to perform observation or the like.

Moreover, with the drive unit 1 arranged near the bending portion of the tube t and the elongated member 40 made relatively short, there is almost no mechanical backlash or a play during operation. So that the bending device A has significantly high operability and is excellent for application for an endoscope device.

Note that, the connecting structure of the front end of the elongated member 40 (structure in which the conical surface receives the spherical engagement part 42) and the connecting structure of the rear end of the elongated member 40 (structure in which the spherical inner surface receives the spherical engagement part 43) are interchangeable, and both the connecting structures may be configured to have the same structure.

Further, any structure allowing rotation and swinging of the elongated member 40 other than the structure illustrated in the drawing can be used in another example of the connecting structure. For example, in a case of the elongated member 40 configured with a twistable and flexible member (e.g., a flexible string), even when one of ends of the elongated member 40 is secured so as not to rotate, the other end of the elongated member 40 twists and deforms to allow rotation and swinging of the elongated member 40.

Further, in the aspect described above, the elongated member 40 is formed of a flexible material. In another example, the elongated member 40 can be formed of rigid materials (e.g., metals and hard synthetic resins). In such example, the operation of advancing the movable shaft 11 to return the tube t to the original shape (linear-tubular shape, etc.) can be performed much faster.

Further, in the aspect described above, the tube t itself is formed of a material having shape retaining property. In another example such as a case where the tube t returns to the original shape by the force received from an external object, where the tube t can easily return to the original shape by forming the elongated member 40 with a rigid material, and where the tube t is used in an application which does not require the tube t to return to the original shape, the tube t may be formed of a flexible material without shape retaining property.

Now, another example according to the present invention will be described. In the example described below, a portion of the Example 1 is modified. So that modified portions will mainly be described in detail. The portion approximately similar to the portion of the Example 1 is appended with the same reference sign, and the description will suitably be omitted to avoid repeated description.

### Embodiment 2

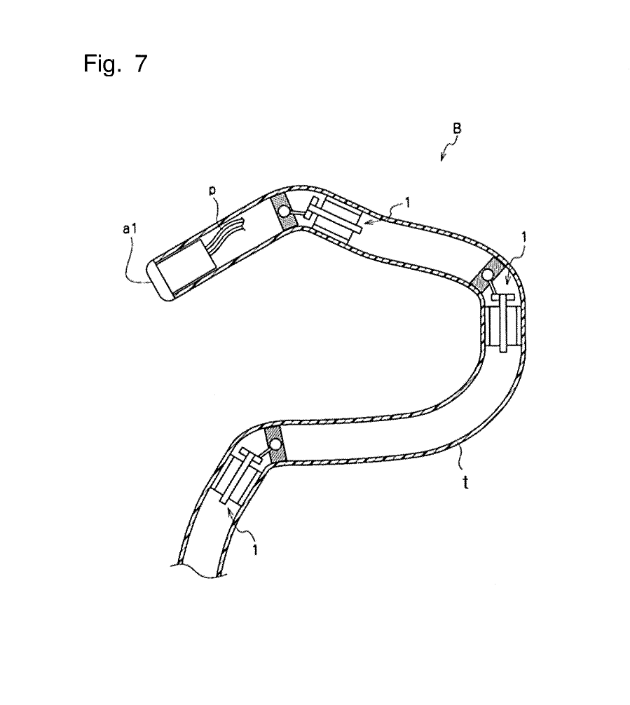

The bending device B illustrated in FIG. 7 is configured to have a plurality of drive units 1 provided, with a gap in the longitudinal direction between each other, in a single tube t so that the tube t can bend at each of a plurality of locations along the longitudinal direction.

The gap between the plurality of drive units 1 is suitably determined according to the application of the bending device B. For example, when the bending device B is used for an endoscope device, as illustrated in FIG. 9, the gap between the adjacent two drive units 1 is determined corresponding to a standard length between corners of a colon (e.g., right and left colic flexures).

A wiring for power supply is independently connected to each of the plurality of drive units 1. Therefore, each of the plurality of drive units 1 can independently be operated.

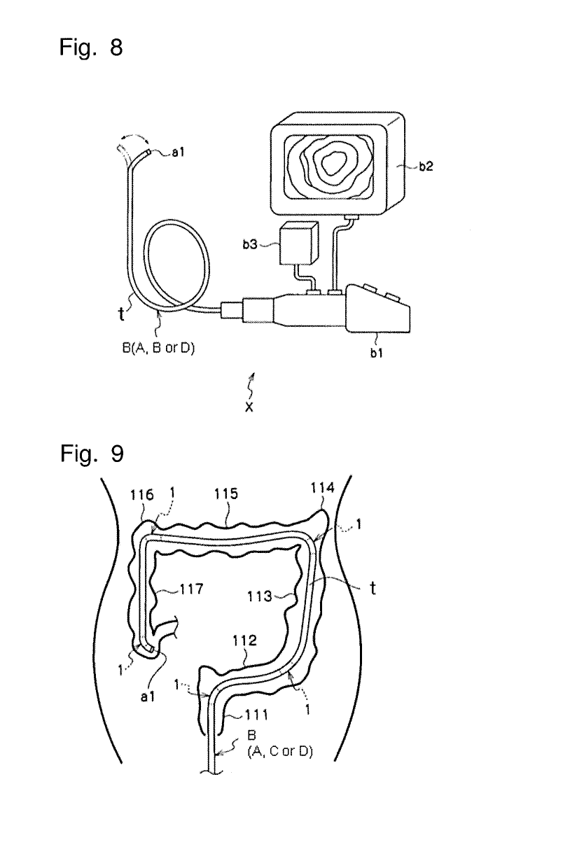

A sensor a1 such as a CCD sensor is attached to the distal end of the tube t of the bending device B as illustrated in the drawing. A wiring p of the sensor a1 is introduced rearward in the tube t through above mentioned penetration holes 51 and 22 of the drive units 1 to be electrically connected to devices (e.g., a controller b1 in FIG. 8) for controlling the bending device B.

FIG. 8 illustrates an endoscope device X configured using the bending device B configured as described above.

The endoscope device X includes the bending device B configured as described above, a controller b1 having a control circuit or switches for controlling the bending device B, a displaying device b2 for displaying an image captured by the sensor a1 on the distal end of the tube t, and a power source device b3 for supplying power to the bending device B.

Now, the effect of the bending device B when the endoscope device X is used for medical application will be described in detail referring to FIG. 9.

As illustrated in the drawing, the bending device B can be inserted in a body with the sensor a1 in the front and reach the ascending colon 117 and the deepest portion of the colon, that is, the portion located further deeper than the right colic flexure 116, by passing through the winding passage with a plurality of corners, that is, the rectum 111, the sigmoid colon 112, the descending colon 113, the left colic flexure 114, the transverse colon 115, the right colic flexure 116, and the like.

Moreover, on inserting the bending device B into the body as described above, the bending device B can be bent along corners of the colon (e.g., the left colic flexure 114 or the right colic flexure 116) by independently controlling each of the plurality of drive units 1 so that the resistance during insertion and irritant and stress to the human body can be reduced. Therefore, the inserting operation can smoothly be performed.

The bending of the plurality of drive units 1 along the corner of the colon can easily be performed by, for example, shooting an X-ray video of the human body to grasp the relative position of each drive unit 1 and the corner during inserting operation.

Further, by controlling the plurality of piezoelectric units 13, each drive unit 1 can bend the tube at any desired circumferential location and also the amount of bend can be changed by any amount. Therefore, by using the bending device B, almost every part of the colon passage can easily be observed and treated and moreover, it may be possible to insert the sensor a1 to reach the small intestine passage connected to the colon.

Further, the endoscope device X configured as described above can be used as an endoscope device to be inserted in other organs such as a gastrofiberscope, or used for an application other than medical application, for example, an industrial endoscope device or the like.

### Embodiment 3

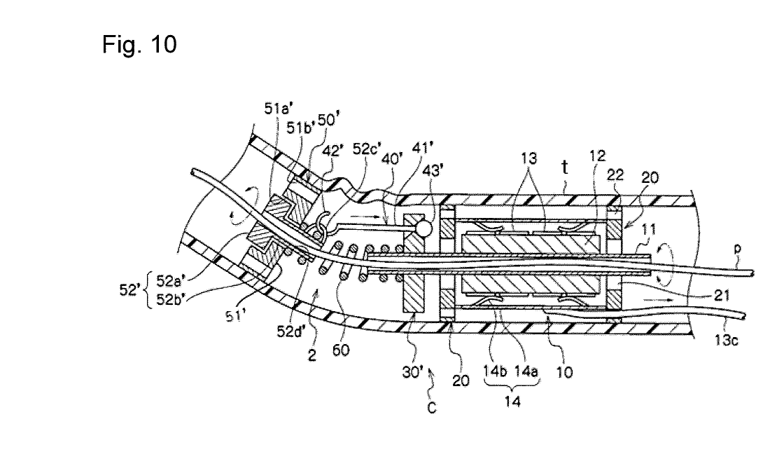

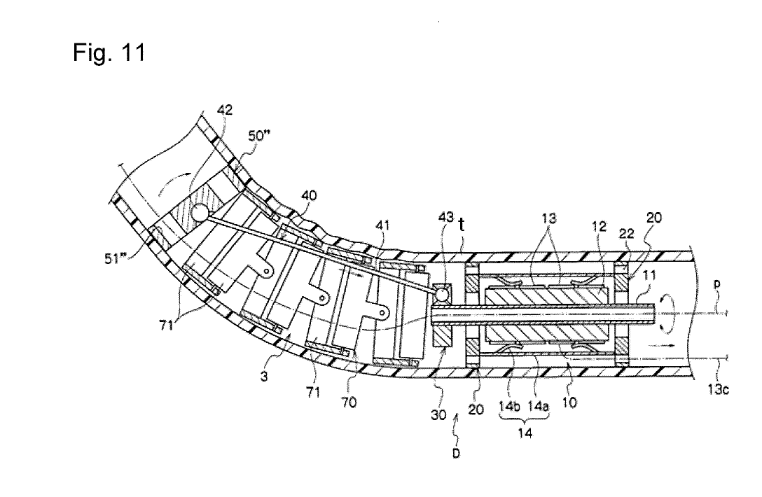

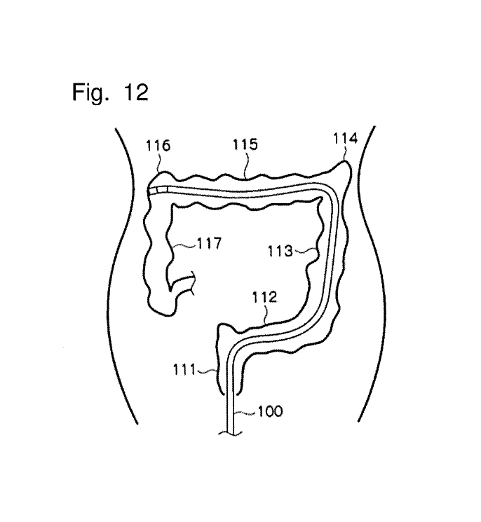

Now a bending device C as illustrated in FIG. 10 will be described.

The bending device C is configured that the drive unit 1 for bending the tube t of the bending device A is replaced with a drive unit 2. The drive unit 2 is configured by modifying the drive unit 1 in a manner that the eccentric connecting member 30 is replaced with an eccentric connecting member 30′, the elongated member 40 is replaced with an elongated member 40′, the connecting member 50 is replaced with a connecting member 50′, and an elastic-sleeve-shaped member 60 (a coil spring, in the example illustrated in the drawing) is added as a recovering member for letting the tube t return to the state before bent.

An eccentric connecting member 30′ has a modified connecting structure for connecting the elongated member 40′. The eccentric connecting member 30′ is basically configured in a manner similar to the eccentric connecting member 30 described above. Approximately similarly to the connecting member 50 (see FIG. 2), the connecting structure of the eccentric connecting member 30′ is configured by rearwardly inserting the main body 41′ of the elongated member 40′ through the eccentric connecting member 30′, and receiving the spherical engagement part 43′ on the rear end of the elongated member 40′ by the conical surface of the eccentric connecting member 30′ so as to allow rotation of the elongated member 40′.

The elongated member 40′ includes a flexible long main body 41′, a spherical engagement part 43′ integrally provided on the rear end of the main body 41′, and an engagement part 42′ integrally provided on the front end of the main body 41′.

The main body 41′ can be, for example, a metal wire, a synthetic resin wire, a string, or the like and has a suitable tensile strength to pull and bend the tube t. Similarly to the bending device A, the main body 41′ may be a rigid long body (a stick made of metal or synthetic resin) or the like.

The engagement part 42′ is formed of metal, synthetic resin, or the like and formed in a shape allowing engagement with a connecting member 50′ which will be described below. The engagement part 42′ is formed in an approximately hook-shape in the example illustrated in the drawing.

The connecting member 50′ is configured with an approximately disk-shaped securing part 51′ secured on the inner circumferential surface of the tube t and a rotating part 52′ rotatably supported in the central portion of the securing part 51′.

The securing part 51′ is connected to the inner circumferential surface of the tube t so as not to advance/retreat nor rotate. The securing part 51′ rotatably supports the rotating part 52′ inserted in a support hole 51a′, having a step and penetrating the securing part 51′, provided in the central portion of the securing part 51′. Further, near the outer periphery of the securing part 51′, a penetration hole 51b′ is provided as required to insert therein a wiring such as a signal line and a power line.

The rotating part 52′ is an approximately sleeve-shaped member that is penetratingly inserted in the central portion of the securing part 51′ and rotatably supported so as not to come off rearward.

To describe in detail, the rotating part 52′ is integrally configured with a cylindrical large-diameter portion 52a′ rotatably engaged with the front side of the connecting member 50′, a cylindrical small-diameter portion 52b′ rearwardly protruding from the large-diameter portion 52a′ to penetrate the connecting member 50′, and an engagement receiving portion 52c′ secured on the portion, in the rear side of the connecting member 50′, of the small-diameter portion 52b′. In the example illustrated in the figure, the engagement receiving portion 52c′ is formed in a ring shape to engage with the engagement part 42′ having a hook shape.

Further, in the large-diameter portion 52a′ and in the small-diameter portion 52b′, a penetration hole 52d′ is provided to insert therein a wiring p such as a signal line and a power line.

The elastic-sleeve-shaped member 60 may be any approximately sleeve-shaped member that can elastically bend in the longitudinal direction and return to the original approximately linear shape. In the example illustrated in the drawing, a coil spring is used. In another example of the elastic-sleeve-shaped member 60, a sleeve-shaped member made of an elastic material such as a rubber can be used.

The front end of the elastic-sleeve-shaped member 60 configured as described above rotatably engages with the rotating part 52′, and the rear end of the elastic-sleeve-shaped member 60 rotatably engages with the movable shaft 11. The wiring p is inserted through the elastic-sleeve-shaped member 60.

The wiring p of the bending device C is an electric cable, an optical fiber cable, or the like for a device other than the drive unit 2 (e.g., the sensor a1, other adjacent drive units 2, etc.). The wiring p is introduced along the longitudinal direction of the tube t through the inside of the connecting member 50′, the elastic-sleeve-shaped member 60, and the movable shaft 11.

Similar to the tube t of the bending device A, a tube having shape retaining property is used as the tube t of the bending device C. However, since the elastic-sleeve-shaped member 60 acts as a recovering member urging the tube t to return to the state before bent, a tube without shape retaining property can be used.

According to the bending device C configured as described above, similarly to the bending device A, the tube t can be bent and straighten throughout the 360-degree range in the circumferential direction as desired by retreating the movable shaft 11 to tow the connecting member 50′ by the elongated member 40′, and by rotating the movable shaft 11 to displace the rear end of the elongated member 40′ along the circumferential direction. The bend angle of the bending portion can also be controlled as desired.

Further, when returning the tube t from the bent state to the original linear-tubular shape, the operation can be performed rapidly because of the effect of the resilience of the elastic-sleeve-shaped member 60.

Moreover, since the wiring p is inserted in the central portion of the tube t, the wiring p will not obstruct the bending of the tube t. That is, if the wiring p is disposed near the outer periphery of the tube t, the inner wall of the tube t and the wiring p interfere when the tube t is bent, which might obstruct the bending of the tube t. But for the bending device C configured as described above, the wiring p is disposed in approximately central portion of the tube t (see FIG. 10) so that such interference is avoided, thereby keeping flexibility of the tube t.

### EXAMPLE 4

Now a bending device D as illustrated in FIG. 11 will be described.

The bending device D is configured that the drive unit 1 of the bending device A is replaced with a drive unit 3. The drive unit 3 is configured by modifying the drive unit 1 in a manner that the connecting member 50 is replaced with a connecting member 50″ and a tubular support member 70 that can deform along the bend of the tube t so as to stay close to, or to contact, the inner circumferential surface of the bent portion of the tube t is added.

The connecting member 50″ has a modified connecting structure for connecting the elongated member 40. The connecting member 50″ is basically configured in a manner similar to the connecting member 50 described above. Approximately similarly to the structure of the connecting portion of the eccentric connecting member 30, the connecting structure of the connecting member 50″ rotatably and swingably holds and supports the spherical engagement part 42 on the front end of the elongated member 40 by the spherical inner surface.

A penetration hole 51″ is provided near the outer periphery of the connecting member 50″ to insert therein the wiring p.

The tubular support member 70 is configured with a plurality of short sleeve-shaped members 71, each of which closely fit against the inner circumferential surface of the tube t, arrayed in the longitudinal direction of the tube t. To a first sleeve-shaped member 71, a second sleeve-shaped member 71 adjacent to the first sleeve-shaped member 71 is connected so as to pivot about a first axis in the radial direction. A third sleeve-shaped member 71 adjacent to the second sleeve-shaped member 71 is connected to the second sleeve-shaped member 71 so as to pivot about a second axis in the radial direction which is circumferentially rotated 90 degrees from the first axis. The plurality of the sleeve-shaped members 71 are connected in series by repeating the connections described above one after another.

In other examples of the tubular support member 70, the tubular support member 70 can be configured as a coil spring provided to stay close to, or to contact, the inner circumferential surface of the tube t, or as a bellows-shaped or a meshed flexible tube or the like provided to stay close to, or to contact, the inner circumferential surface of the tube t.

Further, the wiring p is inserted through the inside of the tubular support member 70. The wiring p is a signal line, a power line, or the like that is introduced in the longitudinal direction of the tube t through the connecting member 50″, the plurality of sleeve-shaped members 71, and the movable shaft 11.

According to the bending device D configured as described above, similarly to the bending device A, the tube t can be bent and straighten throughout the 360-degree range in the circumferential direction as desired by retreating the movable shaft 11 to tow the connecting member 50″ by the elongated member 40, and by rotating the movable shaft 11 to displace the rear end of the elongated member 40 along the circumferential direction. The bend angle of the bending portion can also be controlled as desired.

Moreover, since the tubular support member 70 stays close to the inner circumferential surface of the bending portion, the chances of forming wrinkles or folds on the wall surface in the inner radius side of the bending portion of the tube t can be reduced (see FIG. 11).

In the example, it is configured that the tube t maintains the bent state even after cutting off the electric supply to the drive unit 1. In another example, it can be configured that the elastic resilience returns the tube t to the original state (e.g., linear-tubular shape) when the electric supply to the drive unit 1 is cut off, by making the urging force of the urging member 14b small or omitting the urging member 14b itself.

In the example, the electric actuator 10 for linearly moving and rotating the movable shaft 11 by vibration is used as a particularly preferable specific example. Any electric actuator configured to drive the movable shaft 11 by electric power in the axial direction and in the rotating direction may be used. In other examples, the electric actuator can have a configuration in which a linear motor and a rotating motor are combined, a configuration in which an electromagnetic plunger and a rotating motor are combined, or other configurations.

In the example, as a preferable aspect to improve operability of changing the bending direction of the tube t along the circumferential direction, the front end of the elongated member 40 (or 40′) is connected to the connecting member 50 (50′ or 50″) at a location inwardly distanced in the radial direction from the inner surface of the tube t, and the connecting member 50 (50′ or 50″) is connected to the inner surface of the tube t. In other examples, an aspect in which the front end of the elongated member 40 (or 40′) is directly connected to the inner surface of the tube t can be provided.

Further, the endoscope device X is exemplarily described in an aspect using the bending device B. In other examples, the bending device B can be replaced with any of the bending devices A, C, or D.

The bending device which is an embodiment of the present invention can be used for less invasive operation systems using an endoscope, which are increasingly progressing in recent years. The bending device can bend the tube by electric power to any desired direction by any desired angle using a single drive unit inside the tube. The bending device allows the tube to be made thin so that, together with the high flexibility of the tube, for example, the distalmost portion of a medical endoscope device or the like can be inserted into a deep portion of a winding internal organ. Further, the bending device is useful for applications other than medical endoscope devices, such as industrial endoscope devices or devices for searching inside a tube.

Further, other than the use for an endoscope, the bending device can be used for treatment in the medical field or used for production or the like in the industrial field by implementing a hand of a micro-robot, an actuator, or the like.

### EXPLANATION OF REFERENCE NUMERALS

1, 2, 3: drive unit
10: electric actuator
11: movable shaft
12: vibratable member
13: piezoelectric unit
13a: piezoelectric element
13b: electrode
20: secure bracket
30: eccentric connecting member
40: elongated member
50: connecting member
60: elastic-sleeve-shaped member (recovering member)
70: tubular support member
t: tube
13c, p: wiring
A, B, C, D: bending device
X: endoscope device

What is claimed is:

1. A bending device configured to bend a flexible tube electrically from inside comprising:
an electric actuator configured to drive a movable shaft electrically along a longitudinal direction of the tube in an axial direction and in a rotational direction; and an elongated member of which one of ends is connected to the movable shaft and an other end is connected to an inner surface of the tube at a location distanced, in a longitudinal direction of the tube, from a connecting point connecting the one of ends of the elongated member and the movable shaft, wherein
the connecting point connecting the one of ends of the elongated member and the movable shaft being located eccentric to a center of the movable shaft so that a towing direction of the elongated member towed by retreating the movable shaft changes along a circumferential direction by rotation of the movable shaft.

2. The bending device according to claim 1 comprising:
a connecting member connected to the inner surface of the tube and to the other end of the elongated member, wherein a connecting point connecting the other end of the elongated member and the connecting member is located distanced from the inner surface of the tube.

3. The bending device according to claim 2, wherein a first radius and a second radius are configured to have a dimensional relationship of first radius>second radius, the first radius being an eccentric radius of the connecting point connecting the one of ends of the elongated member and the movable shaft about a center of the movable shaft, and the second radius being an eccentric radius of the connecting point connecting the other end of the elongated member and the connecting member about the center of the movable shaft.

4. The bending device according to claim 2, wherein the elongated member is rotatably and swingably connected to the movable shaft and rotatably and swingably connected to the connecting member.

5. The bending device according to claim 3, wherein the elongated member is rotatably and swingably connected to the movable shaft and rotatably and swingably connected to the connecting member.

6. The bending device according to claim 1, wherein the electric actuator comprises a vibratable member making contact with an outer periphery of the movable shaft; a piezoelectric element secured on the vibratable member; and an electrode secured on the piezoelectric element, and a plurality of sets of the electrodes is arrayed in a circumferential direction and in an axial direction.

7. The bending device according to claim 1, wherein a recovering member for urging the tube to return to a state before bent is provided.

8. The bending device according to claim 1, wherein the elongated member is formed with a flexible long body.
